(19) United States
(12) Patent Application Publication
(10) Pub. No.: US 2004/0001845 A1
(43) Pub. Date: Jan. 1, 2004

(54) Cytotoxic T-cell epitopes of HIV-1 virus

(76) Inventors: Marcus Altfeld, Arlington, MA (US); Xu Yu, Cambridge, MA (US); Bruce D. Walker, Milton, MA (US); Marylyn Addo, Arlington, MA (US)

Correspondence Address: THOMAS POWELL, 7850 CONIFER DRIVE, COLORADO SPRINGS, CO, 80920, US

(21) Appl. No.: 10/442,909

(22) Filed: May 20, 2003

Related U.S. Application Data
(60) Provisional application No. 60/382,120, filed on May 20, 2002.

(51) Int. Cl. A61K 39/21; A61K 39/00

(52) U.S. Cl. 424/185.1; 424/188.1

(57) ABSTRACT

The invention provides compositions containing HIV epitopes, which are recognized by cytotoxic T lymphocytes (CTL). Such polypeptides are used in vaccines and immunotherapies. HIV-1 epitopes represent early targets in a naturally-occurring response against HIV-1 infection.

### PRIORITY INFORMATION

This application claims priority to U.S. Provisional Patent Application Serial No. 60/382,120, filed on May 20, 2002, the entire contents of which is hereby incorporated by reference.

### STATEMENT AS TO FEDERALLY SPONSORED RESEARCH

This invention was made with U.S. Government support under a National Institutes of Health grants R37 AI128568, RO1 AI50429, RO1 AI30914, RO1 AI44656, and RO1 AI40873. The government has certain rights in the invention.

### BACKGROUND OF THE INVENTION

The invention relates to T cell immunity to viral antigens.

Twenty years after the first clinical evidence of acquired immunodeficiency syndrome (AIDS) was reported, more than 60 million people have been infected with HIV-1. AIDS is now the leading cause of death in sub-Saharan Africa and is the fourth-biggest killer worldwide. It is urgent to develop an effective HIV vaccine to either protect from HIV-1 infection or to attenuate the course of disease. Increasing evidence indicates that HIV-1-specific CD8+ cytotoxic T lymphocytes (CTL) and CD4+ T helper cells play a critical role in the control of viral replication in HIV-1 infection, but very little is known about the epitopes involved. Understanding the correlates of protective immunity is a first step in the development of immune-based approaches to control HIV-1 infection.

### SUMMARY OF THE INVENTION

The invention provides compositions containing HIV epitopes, which are recognized by cytotoxic T lymphocytes (CTL) and methods of preventing or attenuating viral infections. Such polypeptides are used in vaccines and immunotherapies. HIV-1 epitopes defined herein represent early targets in a naturally-occurring response against HIV-1 infection. Accordingly, the invention features an immunogenic composition containing an Human Leukocyte Antigen (HLA) class I restricted HIV-1 peptide. The peptide preferably consists essentially of 8-12 amino acids, e.g., 8, 9, or 10 amino acids. A CTL response to the peptide epitope is detected from a week to about a year from initial exposure to the viral pathogen, and may persist as an immunodominant epitope or decrease as the infection progresses. Immunization with the defined epitopes elicits a protective immune response regardless of the stage of infection.

An HIV-1 infection in a mammal, e.g., a human patient, is prevented or the severity of an existing infection reduced by administering to a mammal these immunodominant epitopes. One or more of the HLA class I-restricted epitopes are administered to an HLA-matched individual. Alternatively, a mixture of epitopes (each HLA-restricted to a specific HLA class I allele) is administered as a composite vaccine to protect a genetically diverse (e.g., HLA class I diverse) population from infection. The peptide epitopes are used as a highly immunogenic HIV-1 vaccine.

Accordingly, the invention features an immunogenic composition, which includes an HLA class I restricted HIV-1 polypeptide. Preferably, the polypeptide is less than 200 amino acids in length. More preferably, the length is less than 100, 50, 25, 20, 15 amino acids in length. For example, the polypeptide consists essentially of 8-12 amino acids and is characterized as an early CTL target in HIV-1 infection.

The peptide epitopes described herein associate with HLA class I binding motifs. For example, the immunogenic composition contains an HLA class I A3-restricted HIV-1 polypeptide that contains an amino acid sequence selected from the group consisting of SEQ ID NO: 3, 4, 6, 7, 8, 11, 12, 13, 14, and 15. Preferably, the immunogenic composition contains an HLA class I A3-restricted HIV-1 polypeptide, which consists essentially of an amino acid sequence selected from the group consisting of SEQ ID NO: 3, 4, 6, 7, 8, 11, 12, 13, 14, and 15. Optionally, the composition also contains at least one polypeptide containing an amino acid sequence selected from the group consisting of SEQ ID NO: 1, 2, 5, 9, and 10.

HLA class I B7-restricted HIV-1 polypeptide epitopes include those which contain an amino acid sequence selected from the group consisting of SEQ ID NO:18, 25, and 26. For example, the immunogenic composition contains an HLA class I B7-restricted HIV-1 polypeptide, which consists essentially of an amino acid sequence selected from the group consisting of SEQ ID NO: 18, 25 and 26. The composition further contains at least one polypeptide containing an amino acid sequence selected from the group consisting of SEQ ID NO:16, 17, 19, 20, 21, 22, 23, and 24. An HLA class I Cw7-restricted HIV-1 polypeptide immunogen contains the amino acid sequence of SEQ ID NO:27.

An immunogenic composition contains one or a plurality of HIV polypeptides containing the epitopes described above. For example, a composition contains a first polypeptide which includes an amino acid selected from the group consisting of SEQ ID NO: 3, 4, 6, 7, 8, 11, 12, 13, 14, 15, 18, 25, 26, and 27, and a second polypeptide, which contains an amino acid selected from the group consisting of SEQ ID NO: 1, 2, 5, 9, 10, 16, 17, 19, 20, 21, 22, 23, and 24.

The compositions described herein are used to elicit an HIV-specific immune response. A method of stimulating an HIV-specific immune response is carried out by contacting an immune cell, e.g., a population of effector cells or a mixed population of antigen-presenting cells and effector cells, with an immunogen. The immunogen contains polypeptide that is less than 50 amino acids in length and contains an epitope containing one or more of the following amino acid sequence of SEQ ID NO: 3, 4, 6, 7, 8, 11, 12, 13, 14, 15, 18, 25, 26, and 27. Optionally, the immune cell (or population of immune cells) is further contacted with one or more polypeptides containing an epitope selected from the group consisting of SEQ ID NO: 1, 2, 5, 9, 10, 16, 17, 19, 20, 21, 22, 23, and 24. Preferably, the effector immune cell is a CD8+ T cell. The cells are contacted ex vivo or in vivo. For example, a method of stimulating an HIV-specific immune response is carried out by administering to a mammal an immunogen containing a peptide epitope selected from the group consisting of SEQ ID NO: 3, 4, 6, 7, 8, 11, 12, 13, 14, 15, 18, 25, 26, and 27. Preferably, the mammal is a human. For example, human is a member of a Caucasian race and contains an HLA allele selected from the group consisting of A3, B7, and Cw7. Following immunization with the peptides, an increase in proliferation of HIV-specific CD8+ T cells (compared to the level of proliferation prior to immunization) is detected. In another example, the method is carried out by administering to a member of an Asian race an immunogen containing a peptide epitope selected from the group consisting of SEQ ID NO: 5, 6, 8, 10 and 11. In the latter case, the human subject expresses an HLA A11 allele.

Also within the invention is a method for measuring an immune response in a patient. For example, the method is useful to assess the immune state of an individual prior to commencing treatment and is also useful to monitor the success of a vaccination or treatment regimen. The method is carried out by (a) providing a sample of cytotoxic T lymphocyte effector cells from a patient; (b) providing HLA class I matched detectably-labeled target cells, which have been pulsed with an HIV-1 polypeptide; (c) contacting the effector cells and target cells; and (d) determining the amount of label released by the target cells. An increase in the amount of label released compared to a control value indicates the presence of an HIV-specific immune response in the patient.

The peptide to be administered is a chain of at least four HIV-1 amino acid sequence residues, preferably at least six, more preferably eight or nine, sometimes ten to twelve residues, and usually fewer than about fifty residues, more usually fewer than about thirty-five, and preferably fewer than twenty-five, e.g., eight to seventeen amino acid residues. The sequence is homologous to a corresponding portion of contiguous residues of an HIV-1 protein and contains a CTL-inducing epitope. Preferably, the amino acid sequence of each polypeptide contains at least 8 contiguous amino acids of a naturally-occurring HIV-1 protein. Such peptides and peptide mixtures are useful as vaccines or in immunotherapy approaches for viral infections. A preferred length is 8-10 or 8-12 amino acids.

The polypeptides and nucleic acids described herein are substantially pure. By a substantially pure polypeptide is meant a polypeptide, which is separated from those components (proteins and other naturally-occurring organic molecules) which naturally accompany it. A polypeptide is substantially pure (or purified) when it constitutes at least 60%, by weight, of the protein in the preparation. Preferably, the protein in the preparation is at least 75%, more preferably at least 90%, and most preferably at least 99%, by weight, of the desired peptide. A substantially pure polypeptide is obtained, e.g., by extraction from a natural source; by expression of a recombinant nucleic acid; or by chemically synthesizing the protein. Purity is measured by a number of methods known in the art, e.g., column chromatography, polyacrylamide gel electrophoresis, or HPLC analysis. A protein is substantially free of naturally associated components when it is separated from those contaminants, which accompany it in its natural state. Thus, a protein which is chemically synthesized or produced in a cellular system different from the cell from which it naturally originates is substantially free from its naturally associated components.

In addition to peptides, the invention encompasses nucleic acids, e.g., oligonucleotides, which encode the immunogenic HIV-1 peptide epitopes. The nucleic acids, e.g., DNA or RNA, are substantially pure. By substantially pure DNA is meant DNA that is free of the genes which, in the naturally-occurring genome of the organism from which the DNA of the invention is derived, flank the desired gene sequence. The term therefore includes, for example, a recombinant DNA which is incorporated into a vector, into an autonomously replicating plasmid or virus, or into the genomic DNA of a prokaryote or eukaryote at a site other than its natural site; or which exists as a separate molecule (e.g., a cDNA or a genomic or cDNA fragment produced by PCR or restriction endonuclease digestion) independent of other sequences.

A method of inducing a cytotoxic T cell immune response is carried out by contacting a CTL with an immunogenic HIV-1 peptide epitope to stimulate the CTL to produce cytokines and/or kill a virally-infected cell. The CTL or heterogenous population of cells containing a CTL, e.g., peripheral blood mononuclear cells (PBMC), are contacted with peptide ex vivo or in vivo. For vaccine purposes, the peptide epitope or mixture of peptide epitopes is previously determined, and the peptide and mixture is administered to a wide range of individuals prior to a known exposure to a viral pathogen.

The peptides are prepared synthetically or by recombinant DNA technology. The term peptide is used interchangeably with polypeptide in the present specification to designate a series of amino acids connected one to the other by peptide bonds between the alpha-amino and alpha-carboxy groups of adjacent amino acids. Optionally, one or more peptide bonds are replaced with an alternative type of covalent bond (a “peptide mimetic”) which is not susceptible to cleavage by peptidases. Where proteolytic degradation of the peptides following injection into the subject is a problem, replacement of a particularly sensitive peptide bond with a noncleavable peptide mimetic yields a peptide mimetic, which is more stable and thus more useful as a therapeutic. Such mimetics, and methods of incorporating them into peptides, are well known in the art. Similarly, the replacement of an L-amino acid residue is a standard way of rendering the peptide less sensitive to proteolysis. Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4,-dinitrophenyl. The polypeptides or peptides are either in their neutral (uncharged) forms or in forms which are salts, and either free of modifications such as glycosylation, side chain oxidation, or phosphorylation or containing these modifications, subject to the condition that the modification not destroy the immune stimulatory activity of the polypeptides.

Derivative peptide epitopes have an amino acid sequence, which differs from the amino acid sequence of a naturally-occurring HIV-1 peptide. Such derivative peptides have at least 50% identity compared to a reference sequence of amino acids, e.g., a naturally-occurring HIV-1 peptide. Preferably, a derivative is 90, 95, 98, or 99% identical to a naturally-occurring HIV-1 protein sequence. The derivative contains a conservative amino acid substitution. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu; Asp, Glu; Asn, Gln; Ser, Thr; Lys, Arg; and Phe, Tyr. Nucleotide and amino acid comparisons described herein are carried out using the Lasergene software package (DNASTAR, Inc., Madison, Wis.). The MegAlign module used is the Clustal V method (Higgins et al., 1989, CABIOS 5(2):151-153). The parameter used is gap penalty 10, gap length penalty 10.

Other embodiments and features of the invention will be apparent from the following description thereof, and from the claims.

### BRIEF DESCRIPTION OF THE DRAWINGS

FIG. 1 is a line graph showing viral load in study subject AC-06. Viral loads (in copies HIV-1 RNA per ml plasma) in study subject AC-06 are shown during the 34-month study period in a logarithmic scale. Periods on antiretroviral treatment (HAART) are shaded.

FIG. 2 is a diagram showing relative locations of HIV-1 viral proteins and overlapping peptides to which HIV-1-specific CD8+ T cell responses were detected in subject AC-06. The amino acid sequences and locations within each HIV-1 protein of all overlapping peptides targeted by virus-specific CD8+ T lymphocytes in subject AC-06 are shown. A total of 25 different regions within HIV-1 were targeted by CD8+ T cells.

FIGS. 3A, C, E, and G are bar graphs showing a characterization of the HLA class I restriction. FIGS. 3B, D, F, and H are line graphs showing a characterization of HLA-A3- and HLA-B7-restricted optimal HIV-1-specific CTL epitopes in subject AC06. The optimal amino acid sequence of 2 novel HLA-A3-restricted CTL epitopes and 2 novel HLA-B7-restricted CTL epitopes was determined. HLA restriction was performed by using peptides presented by autologous and partially HLA-matched BCL in a 51 Cr-release assay. In the bar graphs, filled bars represent percentage of specific lysis of target cells pulsed with peptide, cross-hatched bars represent percentage specific lysis of control target cells pulsed with no peptide. Fine-mapping of the optimal epitope was done by using serial dilutions of truncated peptides in an IFN-γ Elispot assay, and results are given as spot forming cells per million PBMC (SFC/Million PBMC).

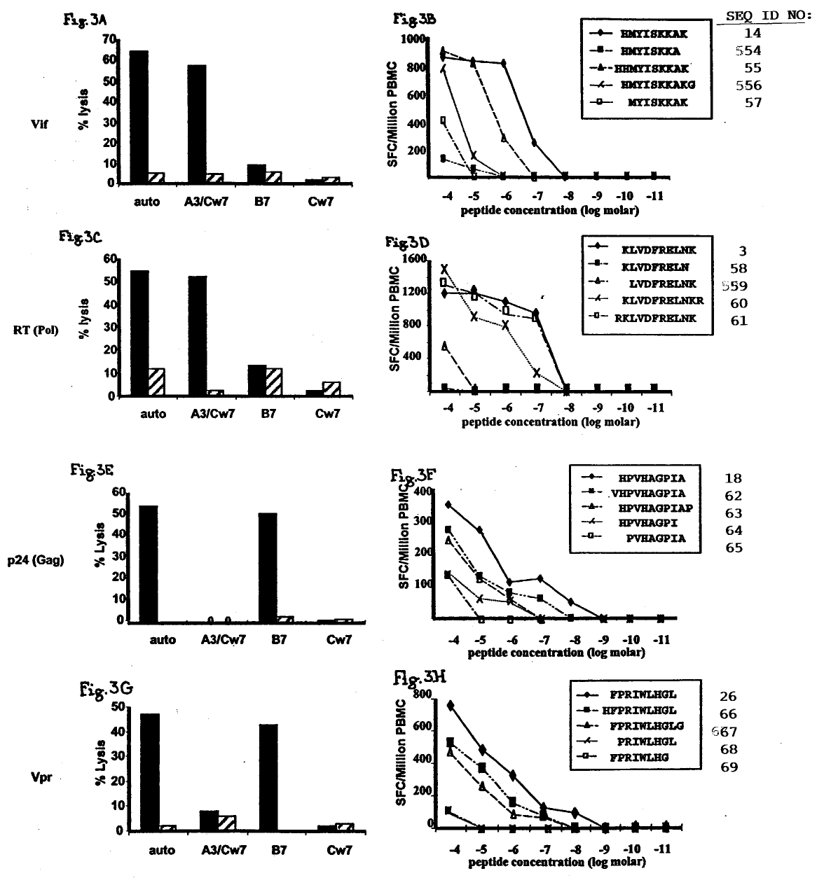

FIG. 4 is a bar graph showing a longitudinal evolution of the magnitude and breadth of HIV-1-specific CD8+ T cell responses on the single epitope level during treated acute infection and supervised treatment interruptions in subject AC-06. The magnitudes of CD8+ T cell responses (given as SFC/Million PBMC) directed against the HIV-1 proteins Gag, Pol, Env, as well as against the accessory HIV-1 proteins (Vif, Vpr and Vpu) and the regulatory HIV-1 proteins (Rev and Tat) are shown during the 34-month study period in subject AC-06. The number of CTL epitopes targeted within each HIV-1 protein or protein group is indicated above each bar. The periods on treatment (HAART) are indicated by the bars on top of the figure, the time after infection and the total number of CTL epitopes targeted at each time point are shown on the bottom of the figure. Only 2 CTL epitopes, one in HIV-1 Gag and on in HIV-1 Env, were targeted during acute infection, but virus-specific CD8+ T cell responses targeted a total of 27 different CTL epitopes at the end of the 2^{nd} supervised treatment interruption.

FIGS. 5A-E are histograms showing a quantification of the contribution of HLA-A3- and HLA-B7/Cw7-restricted CD8+ T cell responses to the total HIV-1-specific CD8+ T cell responses in subject AC-06. PBMC of AC06 were incubated with autologous BCLs pulsed with either the HLA-B7-restricted immunodominant p24 Gag epitope GPGHKARVL (FIG. 5B), all 27 targeted optimal CTL epitopes (FIG. 5C), the 12 HLA-B7/Cw7-restricted optimal CTL epitopes (FIG. 5D) or the 15 HLA-A3-restricted optimal CTL epitopes (FIG. 5E) recognized by AC-06. Non-peptide pulsed BCL were used as negative control (FIG. 5A). The percentage of peptide-specific IFN-γ-producing CD8+ T cells after subtraction of background activity are given in the individual plots.

FIGS. 6A-D are bar graphs showing the frequency of recognition of optimal CTL epitopes during acute HIV-1 infection and after supervised treatment interruption. FIGS. 6E-F are lists of HLA class I restricted epitopes. FIGS. 6A and B show the percentage of individuals expressing HLA-A3 who recognize the individual HLA-A3-restricted optimal CTL epitopes 1 to 15. The amino acid sequences and the corresponding HIV-1 proteins for the CTL epitopes tested are shown at the bottom of the figure. FIG. 6A shows the percentage of individuals expressing HLA-A3 (n=14) who recognize the individual HLA-A3-restricted optimal CTL epitopes during acute HIV-1 infection (filled bars) and after 12 months of treatment with HAART (cross-hatched bars). FIG. 6B shows the percentage of individual expressing HLA-A3 (n=7) who recognize the individual HLA-A3-restricted optimal CTL epitopes following supervised treatment interruptions. FIGS. 6C and D show the percentage of individual expressing HLA-B7 who recognize the individual HLA-B7-restricted optimal CTL epitopes 1 to 15. The amino acid sequences and the corresponding HIV-1 proteins for the CTL epitopes tested are shown at the bottom of the figure. FIG. 6C shows the percentage of individual expressing HLA-B7 (n=11) who recognize the individual HLA-B7-restricted optimal CTL epitopes during acute HIV-1 infection (filled bars) and after 12 months of treatment with HAART (cross-hatched bars). FIG. 6D shows the percentage of individual expressing HLA-B7 (n=7) who recognize the individual HLA-B7-restricted optimal CTL epitopes following supervised treatment interruptions.

### DETAILED DESCRIPTION

Antigen presenting cells (APCs) respond to HIV infection by processing viral fragments and presenting HIV-derived peptides to provoke immune reactivity from CD4+ helper T cells and CD8+ CTLs. When presented, the immunogenic peptides (epitopes) lay within clefts in cell surface HLA molecules. The epitopes are held in the clefts, which are commonly called grooves, by hydrogen bonding between epitope and groove amino acids.

HIV-1-specific CD8+ T cell responses generated during acute infection play a critical role in the control of viremia. However, prior to the invention, little was known about the viral T cell epitopes targeted during acute infection as well as their hierarchy in appearance and relative immunodominance over time. As described below, HIV-1-specific CD8+ T cell responses were characterized in 18 acutely infected individuals expressing HLA-A3 and/or -B7. Detailed analysis of CD8 responses in a person who underwent treatment of acute infection followed by re-exposure to HIV-1 through supervised treatment interruptions (STI) revealed recognition of only two CTL epitopes during symptomatic acute infection. The HIV-1-specific CD8+ T cell responses broadened significantly during subsequent exposure to virus, ultimately targeting 27 distinct CTL epitopes, including 15 different CTL epitopes restricted by a single HLA class I allele (HLA-A3). The same few peptides were consistently targeted in an additional 17 persons expressing HLA-A3 and/or -B7 during acute infection. The data establish a consistent pattern in the development of epitope-specific responses restricted by a single HLA allele during acute HIV-1 infection, and persistence of the initial pattern of immunodominance during subsequent STI. In addition, the data demonstrate that HIV-1-specific CD8+ T cell responses can ultimately target a previously unexpected and unprecedented number of epitopes in a single infected individual, even though these are not detectable during the initial exposure to virus. The epitopes described herein, e.g., those identified as immunodominant during acute infection, are useful in preventative vaccines and therapeutic intervention to inhibit viral replication and reduce viral load in infected individuals. Immunodominant HIV-1-specific CD8+ T cell responses during acute infection were characterized, and CD8+ T cell responses directed against full length HIV-1 comprehensively assessed. These early recognized CTL epitopes represent crucial targets, due to their early presentation, high immunogenicity and involvement on the initial control of viremia. A large panel of 505 overlapping 15-18 mer peptides spanning the full length sequences of HIV-1 lade B was used to comprehensively characterize CD8+ T cell responses during acute HIV-1 infection. The evolution of the magnitude, breadth and hierarchy of CD8+ T cell responses was followed longitudinally in individuals identified and treated during acute HIV-1 infection, and the impact of supervised treatment interruptions (STI) on the evolution and immunodominance of responses was assessed. The data show that a previously unexpected and unprecedented large number of HIV-1-specific CTL epitopes are presented by a single HLA class I allele in an infected individual, and that the temporal appearance of these responses follows similar patterns in persons of the same HLA type with acute HIV-1 infection.

Therapeutic Administration

Polypeptides (or nucleic acids encoding the peptides) described herein are useful to induce HIV-1-specific CTL for the purpose of preventing infection or attenuating an existing infection. When a peptide is used as a vaccine, it is administered to a patient in the form of a peptide solution in a pharmaceutically acceptable carrier. Standard methods for delivery of peptides are used, e.g., for intracellular delivery, the peptides are packaged in liposomes. Such methods are well known to those of ordinary skill in the art. The peptides are administered at an intravenous dosage of approximately 1 to 100 μmoles of the polypeptide per kg of body weight per day. The compositions of the invention are useful for parenteral administration, such as intravenous, subcutaneous, intramuscular, and intraperitoneal. For example, a unit dose of the peptide ranges from 0.1 to 100 mg, which may be administered at one time or repeatedly to a patient. A plurality of peptides are optionally administered together (simultaneously or sequentially). The peptides in the mixture bind to the same or different HLA class I molecules to yield a vaccine that elicits a CTL response in the majority of individuals in a genetically diverse population.

Peptides are recombinantly produced or synthetically made using known methods. Peptide solutions are optionally lyophilized or granulated with a vehicle such as sugar. When the compositions are administered by injection, they are dissolved in distilled water or another pharmaceutically acceptable excipient prior to the injection. The peptides are used to induce a HIV-1-specific CTL response in vivo or ex vivo. The peptides are administered directly to an individual or lymphocytes (e.g., derived from peripheral blood mononuclear cells) are removed from an individual, cultured with one or more peptides, and returned to the individual. For example, 0.01 to 1 mg of the peptide is added to 107 to 109 peripheral blood lymphocytes obtained from a patient, then the cells are cultivated for several hours to one day. The cells are then introduced back into the patient. In such ex vivo therapy, cell stimulation is performed outside the body. For example, lymphocytes or other target cells are removed from a patient and contacted with immunogenic peptides, providing a stimulatory concentration of peptide in the cell medium far in excess of levels which could be accomplished or tolerated by the patient. Following treatment to stimulate the CTLs, the cells are returned to the host to treat a viral infection. The host's cells may also be exposed to vectors which carry nucleic acids encoding the peptides. The ex vivo stimulated cells are returned to the patient after reaching a predetermined cell density.

Alternatively, the cells are continuously cultivated ex vivo in a culture medium to which recombinant interleukin 2 and 1 μg/ml of the peptide has been added. The cells are cultured over several hours, days, or several weeks to induce CTL. Activated ex vivo stimulated CTL are then intravenously injected into the patient.

The peptides or combinations of peptides (multiepitopic vaccine compositions) are selected by and tailored to a target population. Populations are characterized by race and/or expression of HLA alleles. For example, HLA alleles A3 and B7 are common in Caucasian populations. Accordingly, HLA A3-restricted and B7-restricted peptides are used as immunogens for Caucasian populations. The HLA A11 allele is commonly expressed in Asian, e.g., Chinese, populations. Peptides such as those containing the amino acid sequence of SEQ ID NO: 5, 6, 8, 10, or 11 are used as immunogens for Asian populations.

In addition to monoepitopic or multiepitopic peptide immunogens, fragments of the HIV-1 virus spanning regions encoding desired epitopes (selected by HLA allele expression profiles of target populations) are administered. Preferably, the region contains early expressed genes. Regions containing genes encoding nef, rev, vif, and vpr are useful to induce a CTL response.

DNA encoding a peptide epitope may also be administered, e.g., by incorporating the DNA into a viral vector. Nucleic acids are adminstered using known methods, e.g., intravenously, at a dose of approximately 10⁶ to 10²² copies of the nucleic acid molecule.

A pharmaceutical composition contains a pharmaceutically-acceptable excipient and optionally contains a composition, which primes CTL, e.g., a lipid such as tripalmitoyl-S-glycerylcysteinly-seryl-serine. The concentration of CTL stimulatory peptides ranges from less than about 0.1%-1% to as much as 20 to 50% or more by weight, depending on mode of administration selected. Dosage determination and excipient choice is well within the skill of those practicing in the art of medicine and pharmaceuticals.

Vaccine compositions containing immunodominant CTL stimulatory peptides are administered to a patient susceptible to or otherwise at risk of HIV infection to enhance the patient's own immune response capabilities. An immune stimulatory dose is one that increases the level of antigen-specific immunity compared to the level prior to immunization. For example, peptides are administered at a dose of 1.0 μg to about 500 mg per 70 kilogram patient, more commonly from about 50 μg to about 200 mg per 70 kg of body weight. The peptides are administered to individuals of an appropriate HLA type. Alternatively, a mixture of peptides is administered. Individual peptides bind to a specific HLA class I molecules; however, the binding specificity of the mixture encompasses several frequently expressed haplotypes to ensure a broad CTL response in a diverse population of individuals.

Epitopes were evaluated and identified from HIV-1 infected individuals as follows.

Characterization of HIV-1 Epitopes During Acute Infection

Eighteen subjects were evaluated. Study subject AC-06 is an HIV-1 infected individual diagnosed with symptomatic acute HIV-1 infection and treated with highly active antiretroviral therapy (HAART), including two nucleoside analogs and one protease inhibitor, prior to HIV-1 seroconversion. At diagnosis, viral load was 8.1 million copies HIV-1 RNA per ml and CD4+ T cell count was 551 cell/pd. After 18 months of effective antiretroviral treatment, this individual underwent two cycles of supervised treatment interruption (STI). The study protocol required that during STI, treatment had to be reinitiated once viral load rose above 50,000 copies HIV-1-RNA per ml at a single timepoint or remained above 5,000 copies HIV-1-RNA per ml for more than 3 consecutive weeks. The HLA type of this individual was homozygous in the HLA class I A, B and Cw alleles (A3, B7, Cw7), as determined by standard sequence-specific primer PCR.

In addition to study subject AC-06, 17 additional individuals identified during primary HIV-1 infection and expressing either HLA-A3 (n=7), HLA-B7 (n=4) or both alleles (n=6) were studied. Median initial viral load at presentation in these individuals was 750,000 (range 22,000 to 1,100,000) copies HIV-1-RNA per ml. HIV-1 specific CD8+ T cell responses in these individuals were assessed during early HIV-1 infection, as well as after one year of treated infection and following supervised treatment interruptions in a subset of 6 individuals.

The following cell lines and media were used in the analyses. Epstein-Barr virus (EBV)-transformed B-lymphoblastoid cell lines (B-LCL) were established from peripheral blood mononuclear cells (PBMC) and maintained in R20 medium (RPMI 1640 medium (Sigma, St. Louis, Mo) supplemented with 2 mM L-glutamine, 50U of penicillin per ml, 50 μg streptomycin per ml, 10 mM HEPES and 20% heat-inactivated fetal calf serum (Sigma). For culture of CTL clones, medium containing 10% fetal calf serum (R10) supplemented with 50U recombinant interleukin-2 per ml.

HIV-1 peptides were synthesized using standard methods. Five hundred and five overlapping peptides (15-18 mers with 10 amino acid overlap) spanning all the HIV-1 clade B Gag (p15, p17 and p24), Pol (Int, Prot and RT), Env (gp12O and gp4I), regulatory (Rev, Tat) and accessory (Vpr, Vpu, Vif and Nef) protein sequences were synthesized on an automated peptide synthesizer (MBS 396, Advanced ChemTech, Louisville, Ky.) by fluorenylmethoxycarbonyl chemistry. Peptides included those corresponding to optimal HIV-1 CTL epitopes described for the individual's HLA class I type, and truncated peptides for the fine-mapping of novel optimal CTL epitopes.

HIV-1-specific CD8+ T cell responses were characterized by Elispot assay. PBMC were plated out at 100,000 cells per well with peptides at a final concentration of 10-5 molar in 96-well polyvinylidene difluoride-backed plates (MAIP S45; Millipore, Bedford, Mass.) precoated with 0.5 μg/ml anti-1FN-γ mAb, 1-DIK (Mabtech, Stockholm, Sweden) overnight at 4° C. 100,000 PBMC were incubated with R10 alone as negative control and with PHA as positive control. The plates were incubated overnight (14-16h) at 37° C., 5% CO₂ and then processed using known methods, e.g., the method described by Altfeld et al., 2000, J. Virol. 74:8541-8549. The number of specific IFN-γ secreting T-cells was counted by direct visualization and calculated by subtracting the negative control value and expressed as spot forming cells (SFC) per 10⁶ input cells. Negative controls were always <30 SFC per 10⁶ input cells. Results of 60 or more SFC per 10⁶ input cells above background were considered positive. CD8+ T cell dependence of all responses to synthetic peptides was confirmed by CD4+/CD8+ T depletion/enrichment studies using magnetic beads (MACS, Miltenyi Biotech, Germany). Fine-mapping of epitopes was performed by Elispot assay using serial dilutions of truncated peptides. 50,000 to 100,000 freshly isolated PBMC per well were incubated with peptides at concentrations ranging from 10⁻⁴ M to 10⁻¹¹M overnight on the Elispot plate. All assays were run in duplicate. The optimal peptide was defined as the peptide that induced 50% maximal specific IFN-γ production by T-cells at the lowest peptide concentration.

CTL clones were isolated by limiting dilution by standard methods, using the anti-CD3-specific mAb 12F6 as stimulus for T cell proliferation. Developing clones were screened for HIV-1-specific CTL activity by ⁵¹chromium-release assay against autologous B-cell lines pulsed with the peptides recognized in the Elispot assays. HIV-1-specific clones were maintained by stimulation every 14 to 21 days with an anti-CD3 monoclonal antibody and irradiated allogeneic PBMC. HLA-restriction of CTL epitopes was determined using a panel of target cells matched through only one of the HLA-A, HLA-B or HLA-C class I alleles expressed by the effector cells.

Peptide-specific CD8+ T cell lines were generated as follows. CD8+ T cells were nonspecifically expanded from PBMC over 10 days by using a bi-specific anti-CD3/CD4b monoclonal antibody. Peptide-specific CD8+ T cells were subsequently isolated using an IFN-γ capture assay. Briefly, 10×10⁶ CD8+ T cells were incubated with 20 μM peptide and 1 μl each of the antiCD28 and anti-CD49d mAbs (Becton Dickinson) on 24-well plates at 37° C., 5% CO₂, for 6-8 hours. Cells were subsequently labeled with a bi-specific CD45/IFN-γ Catch Reagent and incubated for 45 mm at 37° C., 5% CO₂. After several washes, the IFN-γ producing cells were stained with a second PE-conjugated IFN-γ detection antibody and separated by anti-PE MicroBeads on a MACS separator. The isolated cells were then expanded for 10 days using autologous irradiated feeders.

Antigen-induced intracellular IFN-γ secretion was detected by flow cytometry. Intracellular cytokine staining was carried out as follows: 0.5-10⁶PBMC or CTL lines were incubated with 4 μM of peptide and 1 μg/ml each of the anti-CD28 and anti-CD49d mAbs (Becton Dickinson) at 37° C., 5% CO₂ for 1 hour, before the addition of 10 μg/ml of Brefeldin A (Sigma, St Louis, Mo.). Following a further 6 hours incubation at 37° C., 5% CO₂, the cells were placed at 4° C. overnight. Cells were then washed and stained with surface antibodies, anti-CD8-PerCP, anti-CD4-APC (Becton Dickinson) at 4° C. for 30 minutes. Following washing, the cells were fixed and permeabilized using Caltag Fixation/Permeabilization Kit (Caltag, Burlinghame, Calif.) respectively for 15 minutes at room temperature in the dark and anti-IFN-γ-FITC was added (Becton Dickinson) at 4° C. for 30 minutes. Cells were then washed and analyzed on a FACS Calibur flowcytometer (Becton Dickinson, San Jose, Calif.). Control conditions were established by the use of autologous PBMC, which were treated identically but without the stimulation of peptide. Assays using HLA-matched or mismatched BCL were run for the determination of HLA-restriction of responses using standard methods. BCL that were pulsed with 10 μM peptide for 1 hour were washed four times prior to incubation with effectors at 10⁵ BCL and 4×10⁵ effectors in 1 ml of R10. The anti-CD28 and anti-CD49d mAbs were then added and the assay run as described above.

Statistical analysis and graphical presentation was done using SigmaPlot 5.0 (SPSS Inc., Chicago, Ill.). Results are given as mean±standard deviation (SD) or median with range. Statistical analysis of significance (p-values) was based on two-tailed t-tests.

Comprehensive Characterization of HIV-1-spec:flc CD8+ T Cell Responses During Supervised Treatment Interruptions in Study Subject A C-06

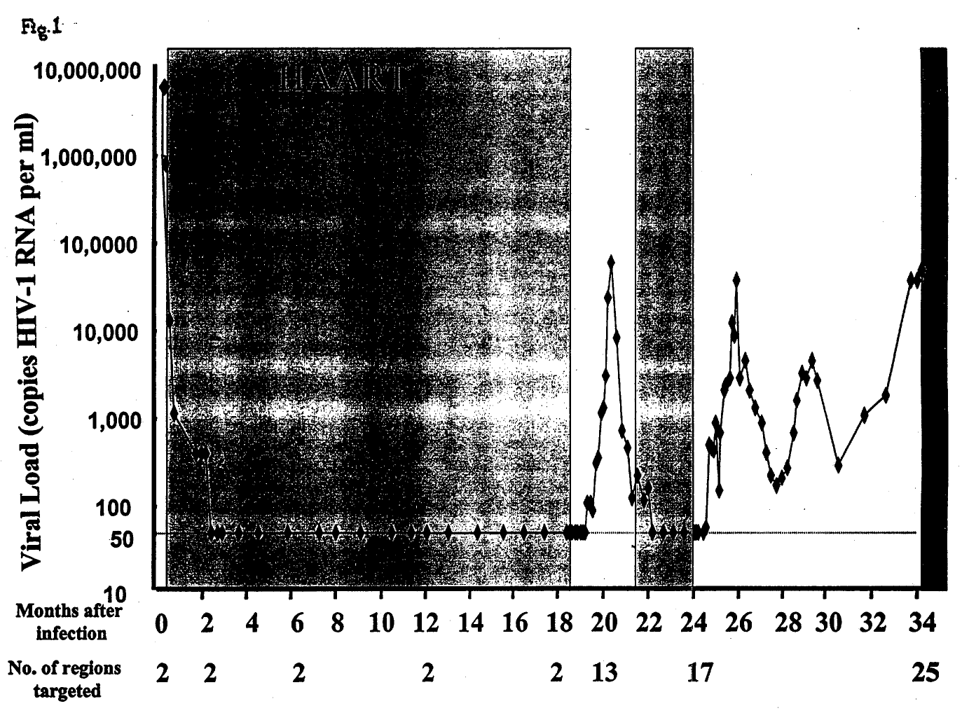

To examine the initial induction of immune responses in HIV-1 infection, a detailed analysis of CD8 responses to all expressed viral proteins in the acute phase of infection was carried out. Early treatment followed by treatment interruption allowed for longitudinal analysis of the subsequent evolution of these responses. Initial studies were performed in subject AC-06 who was diagnosed during symptomatic acute HIV-1 infection with a viral load of 8.1 million copies HIV-1 RNA per ml plasma. Following treatment with HAART prior to HIV-1 seroconversion, viremia dropped to undetectable levels within 6 weeks and remained undetectable for 18 months of treatment prior to the initiation of supervised treatment interruptions. The evolution of viral loads in individual AC-06 during acute HIV-1 infection and two cycles of supervised treatment interruptions are shown in FIG. 1.

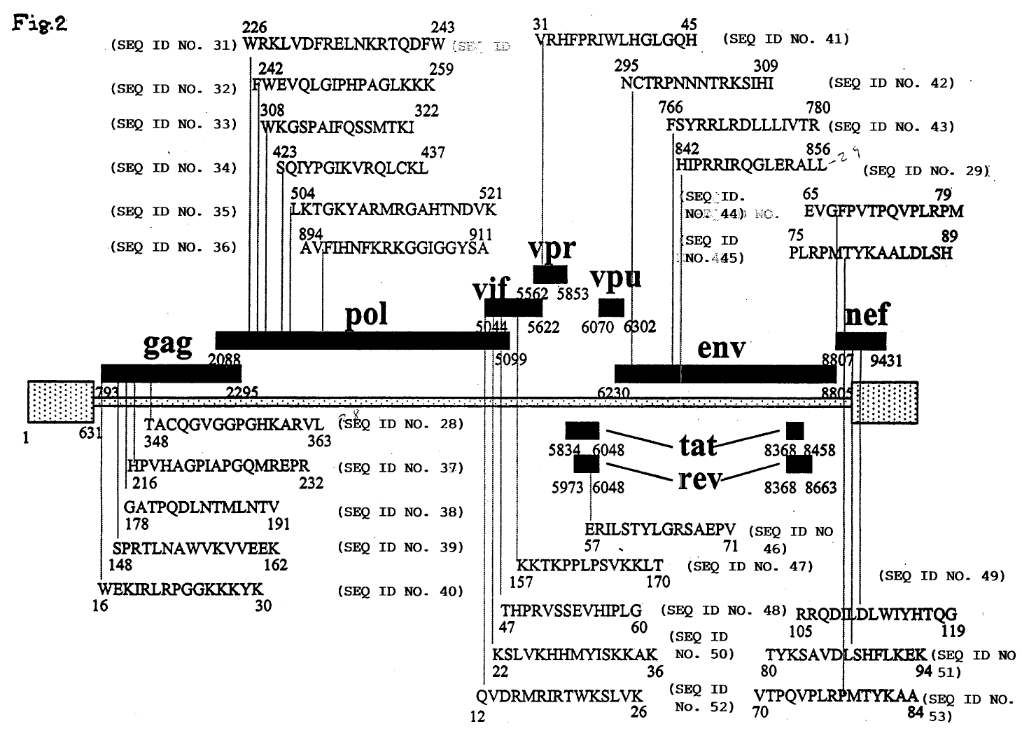

A comprehensive characterization of HIV-1-specific CTL responses was performed in individual AC-06 during the study period of 34 months following acute infection, using a panel of 505 individual overlapping 15-18-mer peptides spanning the full length sequences of HIV-1 lade B by Elispot assay. During acute infection, virus-specific T cell responses were narrowly directed against one peptide in p24 Gag (TACQGVGGPGHKARVL; SEQ ID NO:28) and one peptide in gp41 Env (HIPRRIRQGLERALL; SEQ ID NO:29). The virus-specific T cell responses were enhanced and broadened during re-exposure to antigen, such that at the end of the second STI 34 months after acute infection, subject AC-06 had developed T cell responses against a total of 26 regions of HIV-1 proteins, including 15 responses against structural and ii responses against regulatory and accessory proteins (FIGS. 1 and 2). Except for HIV-1 Tat and Vpu, all HIV-1 proteins were targeted by virus-specific T cells in this individual. All responses were CD8+ T cell mediated, as confirmed by CD4+/CD8+ T cell depletion/enrichment studies, except for one CD4+ T cell mediated response to an overlapping peptide in HIV-1 Integrase (TKELQKQITKIQNFRVYY; SEQ ID NO:30). The magnitude of responses to the individual overlapping peptides ranged from 60 to 1,800 SFC/million PBMC (median 710 SFC/million PBMC). These data show that HIV-1-specific CD8+ T cell responses are narrowly directed during acute HIV-1 infection, despite exposure to enormous levels of plasma virus, but that these responses broaden during re-exposure to lower subsequent levels of viral antigen, ultimately targeting a large variety of different HIV-1 proteins.

Characterization of Optimal CTL Epitopes Targeted by HIV-1-Speciflc CD8+ T Cells

The data describe the regions within HIV-1 targeted by CD8+ T cells in subject AC-06. Studies were undertaken to determine the number of epitopes contained within each of the proteins (e.g. some overlapping peptides may contain the same minimal epitope in their overlap, or a single overlapping peptide may contain more than one epitope) The data showed recognition of a number of epitopes not previously described to be presented by these HLA alleles.

Optimal epitopes as well as restricting HLA alleles were therefore determined. Following a nonspecific polyclonal expansion of PBMC for 10 days using a bi-specific anti-CD3/4b monoclonal antibody, an adapted IFN-γ capture assay was performed to isolate peptide-specific CD8+ T cell lines. Using this approach, 13 different cytotoxic CD8+ T cell lines specific for previously undescribed epitopes were isolated from PBMC. The HLA restriction of these CTL epitopes was determined by ⁵¹chromium-release assay using partially HLA class I matched antigen presenting cell lines (BCLs). Subsequently, the optimal epitope sequences were determined by Elispot assay, using PBMC and serial dilutions of truncated peptides. This approach led to the identification of two novel HLA-A3-restricted and two novel HLA-B7-restricted epitopes shown in Figs. A-H. For one response to a subdominant HIV1 Gag epitope, CD8+ T cell frequencies were too low (80 SFC/Million PBMC) to isolate peptide-specific lines using the IFN-γ capture assay, and epitope-specific CD8+ T cell lines were isolated using standard limiting dilution assays. Overall, 10 novel HLA-A3-restricted, 3 novel HLA-B7-restricted optimal CTL epitopes were defined in subject AC-06, as well as the first HLA-Cw7-restricted CTL epitope described for HIV-1. Fifteen exemplary HLA-A3-restricted epitopes and eleven HLA-B7-restricted epitopes were identified in this individual (Table 1). The newly described epitopes included two epitopes, which were previously reported to be restricted by HLA-A11, an allele closely related to HLA-A3. The majority of the novel epitopes exactly fit HLA-A3, -B7 and -Cw7 peptide binding-motif (Table 1). These results indicate a previously unexpected and unprecedented breadth of virus-specific CD8+ T cell responses, including simultaneous recognition by CD8+ T cells of 15 HLA-A3-restricted, 11 HLA-B7-restricted and 1 HLA-Cw7-restricted optimal HIV-1-specific CTL epitopes.

Longitudinal Evaluation of CTL Responses

Following the characterization of all optimal HIV-1-specific CTL epitopes targeted in subject AC-06, the evolution of HIV-1-specific CD8+ T cell responses over time and the impact of re-exposure to antigen on these responses was addressed. Virus-specific CD8+ T cell responses in this individual were determined at 11 different time points during the 34-month study period, starting from acute HIV-1-infection. This approach allowed assessment of the hierarchy in the appearance of responses to CTL epitopes restricted by the same HLA class I allele over time.

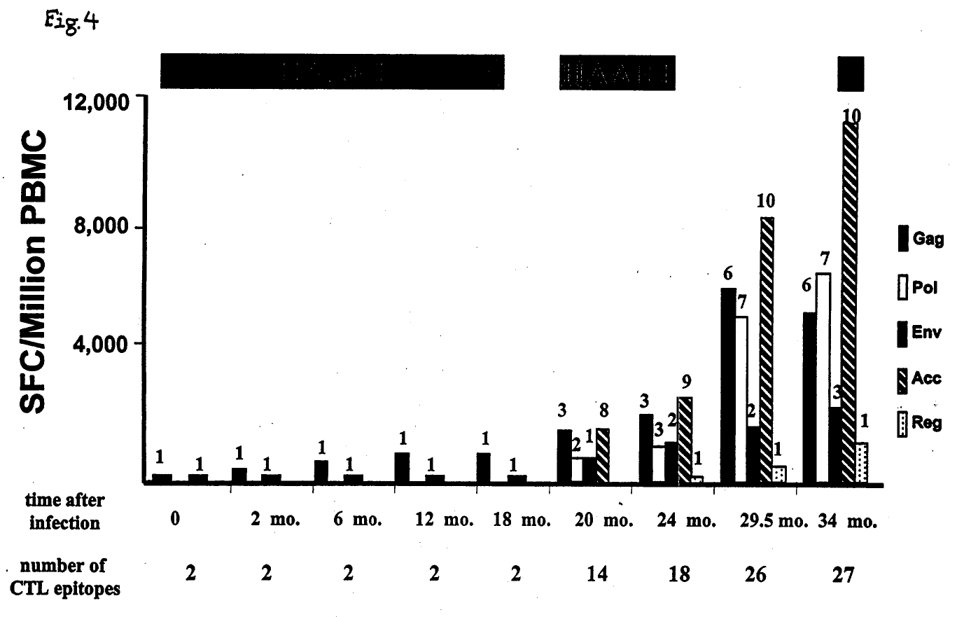

During acute HIV-1 infection, CD8+ T cell responses in subject AC-06 were only directed against two HLA-B7-restricted epitopes in p24 Gag (GPGHKARVL; SEQ ID NO:19) and gp41 Env (IPRRIRQGL; SEQ ID NO:22) (FIG. 4), with no involvement of detectable HLA-A3-restricted epitopes resulting from this initial exposure to virus. During the short first treatment interruption, re-exposure to antigen following viral rebound (see FIG. 1) was accompanied by an enhancement in the magnitude of pre-existing CD8+ T cell responses, as well as by the induction of 12 novel responses to different epitopes (Table 2 and FIG. 4). These included 6 HLA-B7-restricted epitopes, as well as 5 HLA-A3-restricted epitopes and 1 HLA-Cw7-restricted epitope. The initial HLA-B7-restricted p24 Gag epitope GPGHKARVL (SEQ ID NO: 19) remained the immunodominant epitope. The HLA-A3-restricted p17 Gag epitope RLRPGGKKK (SEQ ID NO:2) was targeted for the first time after the first treatment interruption, and evolved to become the immunodominant HLA-A3-restricted epitope at 29.5 months, prior to reinitiation of antiretroviral treatment. Total HIV-1-specific CD8+ T cell responses were augmented from 980 SFC/Million PBMC prior to the first cycle of STI to 5,100 SFC/Million PBMC at the end of the first cycle (p<0.01). These responses were further enhanced in magnitude and breadth during the second treatment interruption, augmenting the total HIV-1-specific CD8+ T cell responses to optimal CTL epitopes from 7,750 SFC/Mill PBMC prior to the beginning of the second cycle of STI to 28,300 SFC/Mill PBMC at the end of the second cycle (p<0.00001). This enhancement of responses was due to the augmentation in the magnitude of responses to previously targeted epitopes, as well as to the development of a total of 9 newly detectable CD8+ T cell responses to optimal epitopes during this second cycle, bringing the total number of CTL epitopes targeted to twenty-seven (Table 2 and FIG. 4).

At the end of the second supervised treatment interruption, CD8+ T cell responses directed against epitopes within the accessory proteins (Vpr, Vif and Nef) dominated the total HIV-1-specific CD8+ T cell responses. Ten responses to different optimal epitopes in these proteins were defined, with a total magnitude of 11,920 SFC/M PBMC (representing 42% of total HP/-1-specific CD8+ T cell responses). CD8+ T cell responses directed against optimal CTL epitopes within HIV-1 Gag and HIV-1 Pol were similar in both breadth and magnitude with 6 responses to Gag (5,680 SFC/M PBMC) and 7 responses to Pol (6,880 SFC/M PBMC). Only 3 responses to optimal CTL epitopes within HIV-1 Env (2,520 SFC/Mill PBMC) and 1 response to HIV-1 Rev (1,300 SFC/Mill PBMC) were detectable, respectively, and no CD8+ T cell activity was detectable against the HIV-1 protein Tat and Vpu.

TABLE 1

Optimal CTL epitopes in AC-06
(novel epitopes in bold)

| | | | | | | | |
|---|---|---|---|---|---|---|---|
| HIV-1 | Optimal | SEQ ID | HLA- | HIV-1 | Optimal | SEQ ID | HLA- |
| protein | epitope | NO: | restriction | protein | epitope | NO: | restriction |

| | | | | | | | | | |
|---|---|---|---|---|---|---|---|---|---|
| A3 binding | 2 C | | | | B7 binding | 123 C | | | |
| motif | L K | | | | motif | P L | | | |
| | V Y | | | | A R | | | | |
| | M F | | | | R K | | | | |
| p17 Gag | KIRLRPGGK | 1 | A3 | A3-KK9 | p24 Gag | SPRTLNAWV | 16 | B7 | B7-SV9 |
| p17 Gag | RLRPGGKKK | 2 | A3 | A3-RK9 | p24 Gag | TPQDLNTML | 17 | B7 | B7-TL9 |
| RT Pol | KLVDFRELNK | 3 | A3 | A3-KK10 | p24 Gag | HPVHAGPIA | 18 | B7 | B7-HA9 |
| RT Pol | GIPHPAGLK | 4 | A3 | A3-GK9 | p24 Gag | GPGHKARVL | 19 | B7 | B7-GL9 |
| RT Pol | AIFQSSMTK | 5 | A3 | A3-ATK9 | RT Pol | SPAIFQSSM | 20 | B7 | B7-SM9 |
| RT Pol | QIYPGIKVR | 6 | A3 | A3-QR9 | gp120 Env | RPNNNTRKSI 21 | B7 | B7-RI10 | |
| RT Pol | RMRGAHTNDVK | 7 | A3 | A3-RK11 | gp41 Env | IPRRIRQGL | 22 | B7 | B7-1L9 |
| Int Pol | AVFIHNFKRK | 8 | A3 | A3-AK10 | Nef | TPQVPLRPM | 23 | B7 | B7-TM9 |
| gp 41 Env | RLRDLLLIVTR | 9 | A3 | A3-RR11 | Nef | RPMTYKAAL | 24 | B7 | B7-RL9 |
| Nef | QVPLRPMTYY | 10 | A3 | A3-QK10 | Vpr | FPRIWLHGL | 25 | B7 | B7-FL9 |
| Nef | AVDLSHFLK | 11 | A3 | A3-ALK9 | Vif | HPRVSSEVHI | 26 | B7 | B7-HT10 |
| Rev | ERILSTYLGR | 12 | A3 | A3-ER10 | Cw7 binding | 23456 C | | | |
| Vif | RIRTWKSLVK | 13 | A3 | A3-RK10 | motif | YPDVV Y | | | |
| Vif | HMYISKKAK | 14 | A3 | A3-HK9 | | PGEYI F | | | |
| Vif | KTKPPLPSVKK | 15 | A3 | A3-KK11 | | RAVIL | | | |
| | | | | | Nef | RRQDILDLWIY | 27 | Cw7 | Cw7-RY11 |

TABLE 2
Longitudinal evolution of the magnitude of HIV-1-specific
CD8+ T cell responses at the single epitope level
(given as SFC/Million PBMC)

| Months after | | | | | | | | | | |
|---|---|---|---|---|---|---|---|---|---|---|
| Infection | | 0 | 2 | 6 | 12 | 18 | 20 | 24 | 29.5 | 34 |
| Gag | A3-RK9 | 0 | 0 | 0 | 0 | 0 | 420 | 590 | 1600 | 1100 |
| | A3-KK9 | 0 | 0 | 0 | 0 | 0 | 220 | 370 | 1400 | 1000 |
| | B7-SV9 | 0 | 0 | 0 | 0 | 0 | 0 | 0 | 680 | 120 |
| | B7-TL9 | 0 | 0 | 0 | 0 | 0 | 0 | 0 | 740 | 1300 |
| | B7-HA9 | 0 | 0 | 0 | 0 | 0 | 0 | 0 | 700 | 360 |
| | B7-GL9 | 320 | 380 | 680 | 870 | 900 | 1000 | 1200 | 1300 | 1800 |
| Pol | A3-KK10 | 0 | 0 | 0 | 0 | 0 | 0 | 70 | 1200 | 1800 |
| | A3-GK9 | 0 | 0 | 0 | 0 | 0 | 0 | 0 | 240 | 1800 |
| | B7-SM9 | 0 | 0 | 0 | 0 | 0 | 90 | 130 | 1400 | 1000 |
| | A3-ATK9 | 0 | 0 | 0 | 0 | 0 | 0 | 0 | 290 | 420 |
| | A3-QR9 | 0 | 0 | 0 | 0 | 0 | 760 | 1000 | 1200 | 840 |
| | A3-RK11 | 0 | 0 | 0 | 0 | 0 | 0 | 0 | 840 | 900 |
| | A3-AK10 | 0 | 0 | 0 | 0 | 0 | 0 | 0 | 260 | 120 |
| Env | B7-IL9 | 60 | 60 | 60 | 70 | 80 | 840 | 1000 | 1600 | 1400 |
| | A3-RR11 | 0 | 0 | 0 | 0 | 0 | 0 | 260 | 230 | 320 |
| | B7-RI10 | 0 | 0 | 0 | 0 | 0 | 0 | 0 | 0 | 800 |
| Acc | B7-TM9 | 0 | 0 | 0 | 0 | 0 | 150 | 200 | 320 | 1300 |
| | A3-QK10 | 0 | 0 | 0 | 0 | 0 | 110 | 70 | 1100 | 1000 |
| | B7-RL9 | 0 | 0 | 0 | 0 | 0 | 120 | 180 | 600 | 360 |
| | A3-ALK9 | 0 | 0 | 0 | 0 | 0 | 450 | 350 | 1200 | 1100 |
| | Cw7-RY11 | 0 | 0 | 0 | 0 | 0 | 0 | 0 | 720 | 1000 |
| | B7-FL9 | 0 | 0 | 0 | 0 | 0 | 0 | 210 | 380 | 960 |
| | B7-HI10 | 0 | 0 | 0 | 0 | 0 | 500 | 880 | 1800 | 1800 |
| | A3-RK10 | 0 | 0 | 0 | 0 | 0 | 180 | 270 | 1100 | 1600 |
| | A3-HK9 | 0 | 0 | 0 | 0 | 0 | 110 | 320 | 1000 | 1600 |
| | A3-KK11 | 0 | 0 | 0 | 0 | 0 | 150 | 340 | 600 | 1200 |
| Reg | A3-ER10 | 0 | 0 | 0 | 0 | 0 | 0 | 310 | 460 | 1300 |
| Total | | 380 | 440 | 740 | 940 | 980 | 5100 | 7750 | 22960 | 28300 |

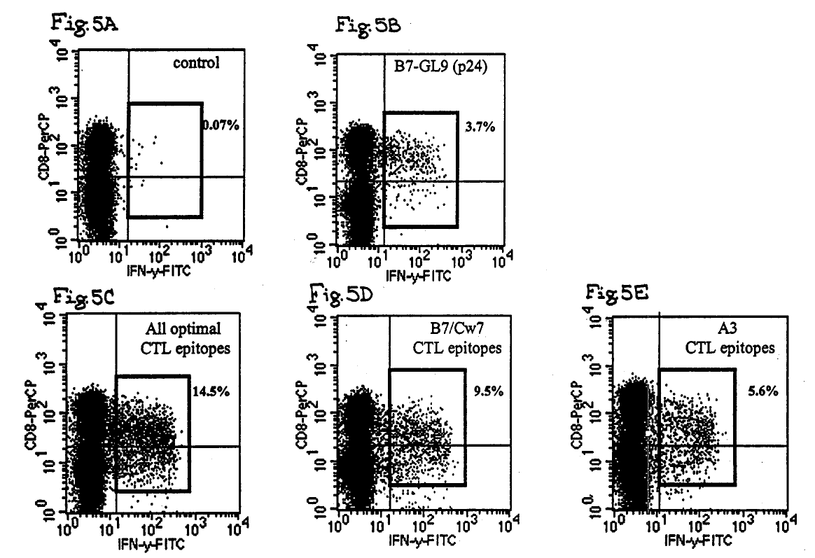

In addition to the IFN-γ Elispot assays, intracellular IFN-γ quantification was measured by flow-cytometry to more accurately quantify the contribution of HLA-A3- and HLA-B7/Cw7-restricted CD8+ T cell responses to the total HIV-1-specific CD8+ T cell responses. Autologous BCL were pulsed with pools of peptides containing either the 15 HLA-A3-or the 12 HLA-B7/Cw7-restricted optimal HIV-1 CTL epitopes targeted by AC-06, or all 27 epitope peptides. A total of 14.5% of CD8+ T cells were specific for the tested epitopes at the end of the second treatment interruption (FIG. 5C). Virus-specific responses were dominated by HLA-B7/Cw7-restricted CD8+ T cell responses (9.5%), contributing more than 60% to total HIV-1-specific CD8+CD8+ T cell responses (FIG. 5D). The p24 Gag epitope GPGHKARVL (SEQ ID NO: 19), the first epitope targeted during acute infection, induced peptide-specific IFN-γ production of 3.7% of CD8+ T cells, contributing more than 24% to the total virus-specific responses (FIG. 5B). These data demonstrate on the single epitope level that HIV-1-specific CD8+ T cell responses were sequentially enhanced and broadened by re-exposure to antigen and ultimately target a previously unexpected high number of 15 different CTL epitopes restricted by a single HLA class I allele in an infected individual homozygous for the HLA class I A, B and Cw alleles. They also indicate that the first targeted HIV-1 epitope during acute HIV-1 infection (GPGHKARVL; SEQ ID NO:19) remains the immunodominant epitope during the broadening of virus-specific CD8+ T cell responses following STI.

Hierarchy of HLA-A3- and HLA-B 7-restricted CD8+ T Cell Responses During Acute HIV-1 Infection and After Supervised Treatment Interruptions

The studies in subject AC-06 demonstrate that only a limited number of HIV-1-specific CD8+ T cell epitopes were detectable in acute infection and additional responses appear following the re-exposure to viral antigen. Experiments were carried out to determine whether the appearance and hierarchy of responses to CTL epitopes restricted by a particular HLA class I allele follows a common pattern in individuals expressing the same allele, using a cohort of 17 additional individuals diagnosed and treated during acute HIV-1 infection and expressing either HLA-A3 (n=7) or HLA-B7 (n=4), or both (n=6). HIV-1-specific CD8+ T cell responses directed against all optimal CTL epitopes described for the HLA class I type of the study subjects, including all newly defined HLA-A3 and -B7 CTL epitopes, were tested in these 18 individuals (including study subject AC-06) during acute infection, as well as after one year of treatment with HAART. In addition to subject AC06, HIV-1-specific CD8+ T cell responses were studied after re-exposure to viral antigen during one cycle of supervised treatment interruptions in a subset of 6 additional individuals (3 expressing A3 and 3 expressing A3 and B7).

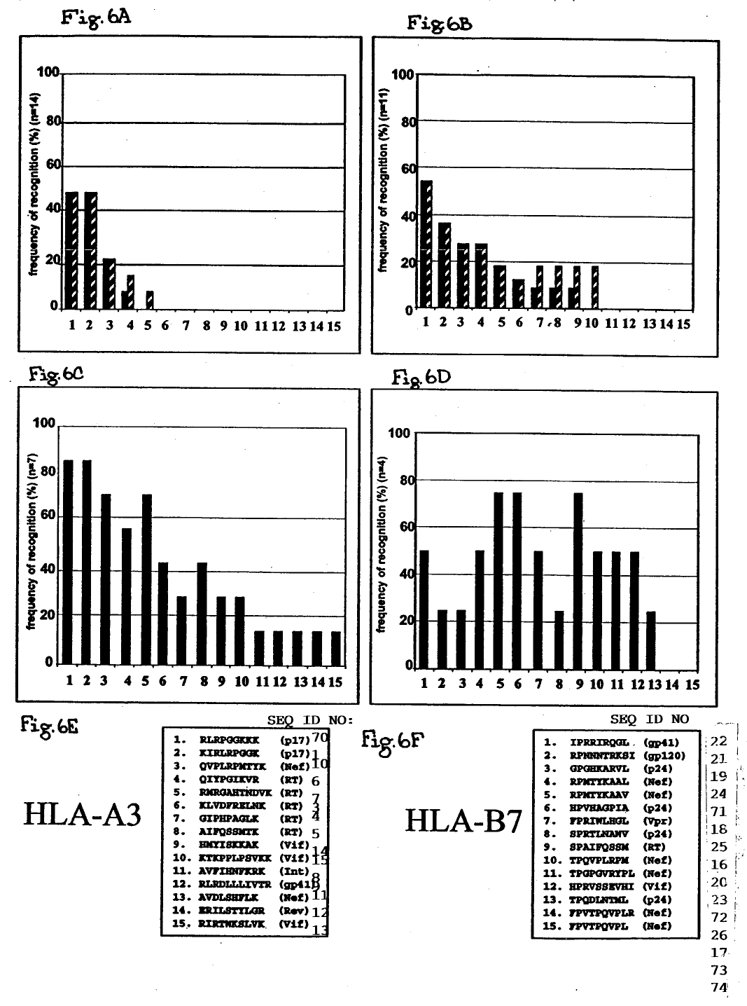

Eight out of the 14 individuals expressing HLA-A3 had detectable HIV-1-specific CD8+ T cell responses restricted by HLA-A3 during acute infection. Only 5 out of 15 HLA-A3-restricted CTL epitopes tested were targeted following acute HIV-1 infection in these individuals (FIG. 6A). Of the HLA-A3-restricted epitopes, two epitopes within p17 Gag (p17-RK9 and p17-KK9) were targeted by 50% (7/14) of the individuals tested (FIG. 6A), representing 88% (⅞) of the individuals with detectable HLA-A3-restricted responses during acute infection. The p17 Gag epitope RLRPGGKKK (SEQ ID NO:2) was the immunodominant A3-restricted CTL response during acute infection. Both p17 Gag epitopes remained the most frequently targeted HLA-A3-restricted CTL epitopes after one year of treatment with HAART (FIG. 6A), as well as after re-exposure following supervised treatment interruption in the 7 studied individuals (FIG. 6B). In addition, in the 4/7 individuals with no detectable HLA-A3-restricted responses during acute infection, the two p17 Gag epitopes RLRPGGKKK (SEQ ID NO:2) and KIRLRPGGK (SEQ ID NO: 1) represented again the first targeted HLA-A3 epitopes, as described in subject AC-06 above. Following re-exposure to antigen, all 15 HLA-A3-restricted CTL epitopes were targeted by at least one individual. However, 5 epitopes were recognized only by subject AC-06, but not by the other individuals expressing HLA-A3 (FIG. 6B and Table 1).

All 11 individuals expressing HLA-B7 had detectable HIV-1-specific CD8+ T cell responses during acute HIV-1 infection. Within the HLA-B7-restricted epitopes, 2 epitopes (gp41-IPRRIRQGL (SEQ ID NO:22) and gp120-RPNNNTRKSI (SEQ ID NO:21)) were targeted by more than 50% (6/11) and 36% (4/11) of individuals with acute infection, respectively (FIG. 6C). However, B7-restricted responses were directed against a larger variety of different epitopes (10 out of 15 tested epitopes) following acute infection. Following supervised treatment interruptions in 4 individuals expressing HLA-B7, the majority (13/15) of described HLA-B7 CTL epitopes were targeted by at least one individual (FIG. 6D).

The earliest targeted CTL epitope B7-GPGHKARVL (SEQ ID NO:19) in p24 Gag remained the immunodominant CTL epitope during the entire study period for study subject AC-06 (FIGS. 5A-E). These studies were extended in the ⅚ individuals who had detectable CD8+ T cell responses during acute infection and for whom follow-up samples during STI were available. In the sixth individual, no CD8+ T cell responses against optimal CTL epitopes were detectable during acute infection. HIV-1-specific CD8+ T cell responses broadened in all individuals following re-exposure to antigen during supervised treatment interruptions. Nevertheless, in all five study subjects the immunodominant CD8+ T cell response during acute infection remained the dominant response at the last available timepoint following re-exposure to antigen. In ⅗ individuals, this response was restricted by HLA-A3, in the other 2 individuals by HLAB8 and HLA-B60, respectively. In addition, the p17-RLRPGGKKK (SEQ ID NO:2) epitope remained the immunodominant HLA-A3-restricted responses in all ⅚ individuals targeting this epitope after treatment interruptions. These data indicate that a subset of epitopes is preferentially targeted during acute HIV-1 infection. These early targeted epitopes remain immunodominant during subsequent enhancement of HIV-1-specific CD8+ T cell responses following re-exposure to viral antigen and are critical epitopes for development of an effective CTL response against HIV-1 infection.

Epitope Targetting

Data indicate that the antiviral immune responses generated during acute infection are crucial for initial control of viral replication and the outcome of chronic infection. The epitopes defined herein play a role in reducing the severity of an HIV-1 infection and protecting an individual from infection.

Prior to the invention, very little was known about the specificity of CTL epitopes targeted during acute HIV-1 infection and the evolution of these epitope-specific responses during later stages of infection. The data described herein reveal the hierarchy in development of epitope-specific CD8+ T cell responses restricted by selected HLA class I alleles and their immunodominance during acute HIV-1 infection. The evolution of these epitope-specific CD8+ T cell responses was also monitored longitudinally during supervised treatment interruptions. The data indicate that for selected HLA alleles the hierarchy in immunodominance of epitope-specific CD8+ T cell responses is determined early during acute infection, and that this hierarchy is little modified during subsequent broadening of responses following re-exposure to antigen. These early treatment studies also show that the initial high level viremia does not induce detectable broadly directed responses, but that these become detectable following repeated exposure to much lower levels of viral antigen, ultimately targeting an unprecedented number of HIV-1-specific CTL epitopes restricted by a single HLA class I allele.

Comprehensive Analysis Identifies CTL Epitope Targets

IFN-γ Elispot assay and flow-cytometry-based assays were used to comprehensively assess HIV-1-specific CD8+ T cell responses in an infected individual identified during acute infection, using a panel of 505 overlapping peptides spanning the full length sequence of HIV-1. The regions within HIV-1 recognized by CD8+ T cells were determined and all optimal CTL epitopes targeted as well as their restricting HLA class I alleles were characterized. Longitudinal studies demonstrated that only two CTL epitopes were targeted by HIV-1-specific CD8+ T cells during acute infection, but that CD8+ T cell responses broadened significantly during subsequent exposure to antigen. Following two cycles of supervised treatment interruptions, 27 distinct CTL epitopes were ultimately recognized by virus-specific CD8+ T cells of the study subject, including 14 novel epitopes (10 of them restricted by HLA-A3). All novel HLA-A3-, HLA-B7- and HLA-Cw7-restricted CTL epitopes fit the peptide-binding motif for the corresponding HLA class I allele. The large number of reported novel HLA-A3-restricted epitopes is surprising in view of the observation that only 8 CTL epitopes restricted by HLA-A3 were described previously prior to the invention.

These data indicate that numerous epitopes are simultaneously presented by a single HLA class I allele. Fifteen distinct CTL epitopes were restricted by HLA-A3 in one individual, including two epitopes previously reported as being restricted by HLA-A11, a closely related HLA class I allele belonging to the HLA-A3-superfamily. In addition, 11 distinct epitopes were restricted by HLA-B7 and one by HLA-Cw7. Several factors may have contributed to this unprecedented breadth of detectable CTL responses. For example, this individual represented the first HIV-1-Infected person, in whom HIV-1-specific CD8+ T cell responses were comprehensively assessed on the single epitope level, including responses directed against all HIV-1 proteins. In addition, the study subject was homozygous for the HLA class I A, B and Cw locus. Therefore, the presentation of viral antigen was necessarily limited to these three HLA class I alleles. In contrast, in heterozygous individuals expressing 6 different major HLA class I alleles, a broader variety of differently restricted epitopes might be presented. Furthermore, different HLA class I molecules may compete with one another for binding and presentation of peptide epitopes. Data from the SIV/macaque model have shown that an animal can recognize simultaneously up to 15 different epitopes restricted by a single MHC class I molecule, and preliminary data on additional HIV-1-infected individuals screened comprehensively for all expressed HIV-1 proteins using overlapping peptides demonstrate that up to 42 different regions within the virus can be targeted in a single infected individual heterozygous for all 3 major HLA class I alleles.

During acute HIV-1 infection, only two CTL epitopes were targeted the subject studied in most detail (AC-06), including the immunodominant HLA-B7-restricted epitope GPGHKARVL (SEQ ID NO: 19) in p24 Gag. Interestingly, this earliest targeted CTL epitope remained the immunodominant response throughout the entire study period, even as virus-specific CD8+ T cell responses broadened significantly to a total of 27 epitopes targeted. These studies were extended to another 17 individuals with acute HIV-1 infection expressing HLA-A3 and/or HLA-B7. For HLA-A3, an immunodominant CTL epitope within HIV-1 p17 Gag was identified (RLRPGGKKK; SEQ ID NO:2). This epitope was targeted during acute infection by 88% of HLA-A3-expressing individuals with detectable CD8+ T cell responses restricted by this allele. In addition, RLRPGGKKK (SEQ ID NO:2) also represented the first HLA-A3-restricted epitope targeted during re-exposure to virus in those individuals who lacked detectable HLA-A3-restricted CD8+ T cell responses during acute infection. In contrast, HLA-B7-restricted CD8+ T cell responses were directed against a larger variety of different epitopes, including the early targeted p24 Gag epitope GPGHKARVL described in AC-06, but also 9 additional epitopes within different HIV-1 proteins. Differences in the CTL epitopes restricted by HLA-A2 have also been noted in acute and chronic HIV-1 infection. These data indicate that immunodominant viral epitopes consistently targeted during acute HIV-1 infection were for specific for certain HLA class I alleles, but that differences in the pattern of development of epitope-specific CTL responses exist among different HLA class I alleles in HIV-1 infection.

Following the re-exposure to antigen during treatment interruptions, CD8+ T cell responses to novel epitopes became detectable in all individuals. These responses represent either newly primed specificities or pre-existing low-frequency responses that are boosted to detectable levels. However, the immunodominant CD8+ T cell response identified during acute infection remained immunodominant after treatment interruptions in all individuals studied. The data demonstrate a consistent pattern in the development and immunodominance of epitope-specific CD8+ T cell responses and indicate that the immunodominance and hierarchy of virus-specific CD8+ T cell responses restricted by HLA-A3 are determined early in HIV-1 infection and persist during re-exposure to viral antigen later in infection. This early determination of epitope-hierarchy during acute HIV-1 infection indicates that the majority of protective antigen-specific CD8+ T cell responses are primed during the initial phase of infection. Some immunodominant epitopes targeted during acute infection have been shown to escape. Responses to these epitopes subsequently decrease.

The data reported herein represents the first comprehensive characterization of HIV-1-specific CD8+ T cell responses in an acutely infected individual, assessing responses directed against all expressed HIV-1 proteins. Detectable virus-specific CD8+ T cell responses were observed to be narrowly directed against a limited number of CTL epitopes during acute HIV-1 infection, despite exposure to enormous levels of viral antigen. These responses can be sequentially enhanced in magnitude and breadth during re-exposure to viral antigen, reaching a previously unprecedented number of targeted optimal CTL epitopes. The temporal hierarchy in the development of epitope-specific responses restricted by HLA-A3 and their immunodominance during acute infection and subsequent treatment interruptions followed similar patterns in the individuals studied. These data indicate consistent early targeting of specific immunodominant epitopes, e.g., by administering the epitopes identified in acute infection and described herein, is important in achieving a highly immunogenic HIV-1 vaccine.

Selection of Peptide Epitopes Based on Target Populations

Optimal epitopes were determined based on an HLA-restriction profile common to a caucasian population (HLA alleles A3, B7, Cw7) Table 1). The peptides were tested for association with HLA allele A11. Five epitopes (SEQ ID NO: 5, 6, 8, 10, and 11) were recognized by individuals expressing HLA-A11. These epitopes represent useful immunogens for asian (e.g., Chinese) populations.

TABLE 3

| HIV | | | RESTRIC- | | |
|---|---|---|---|---|---|
| PROTEIN | EPITOPE | HLA | TION | HLA | |
| P17 Gag | KJRLRPGGK | A3 | A3-KK9 | | SEQ ID NO: 1 |
| P17 Gag | RLRPGGKKK | A3 | A3-RK9 | | SEQ ID NO: 2 |
| RT Pol | KLVDFRELNK | A3 | A3-KK10 | | SEQ ID NO: 3 |
| RT Pol | GIPHPAGLK | A3 | A3-GK9 | | SEQ ID NO: 4 |
| RT Pol | AIFQSSMTK | A3 | A3-ATK9 | All | SEQ ID NO: 5 |
| RT Pol | QIYPGIKVR | A3 | A3-QR9 | All | SEQ ID NO: 6 |
| RT Pol | RMRGAHTNDVK | A3 | A3-RKA11 | | SEQ ID NO: 7 |
| Int | AVFIHNFKRK | A3 | A3-AK10 | All | SEQ ID NO. 8 |
| Gp4 | RLRDLLLIVTR | A3 | A3-RR11 | | SEQ ID NO. 9 |
| Nef | QVPLRPMTYK | A3 | A3-QK10 | All | SEQ ID NO: 10 |
| Nef | AVDLSHFLK | A3 | A3-ALK9 | All | SEQ ID NO: 11 |
| Rev | ERILSTYLGR | A3 | A3-ER10 | | SEQ ID NO: 12 |
| VIF | RTRTWKSLVK | A3 | A3-RK10 | | SEQ ID NO: 13 |
| VIF | HMYISKKAK | A3 | A3-HK9 | | SEQ ID NO: 14 |
| VIF | KTKIPPLPSVKK | A3 | A3-KK11 | | SEQ ID NO: 15 |

Other embodiments are within the following claims.

What is claimed is:

1. An immunogenic composition comprising an HLA class I restricted HIV-1 polypeptide, wherein said polypeptide consists essentially of 8-12 amino acids and wherein said peptide is characterized as an early CTL target in HIV-1 infection.

2. An immunogenic composition comprising an HLA class I A3-restricted HIV-1 polypeptide, said polypeptide comprising less than 50 amino acids in length, wherein the amino acid sequence of said polypeptide comprises an amino acid sequence selected from the group consisting of SEQ ID NO: 3, 4, 6, 7, 8, 11, 12, 13, 14, and 15.

3. An immunogenic composition comprising an HLA class I A3-restricted HIV-1 polypeptide, wherein said polypeptide consists essentially of an amino acid sequence selected from the group consisting of SEQ ID NO: 3, 4, 6, 7, 8, 11, 12, 13, 14, and 15.

4. The composition of claim 2, wherein said composition further comprises at least one polypeptide comprising an amino acid sequence selected from the group consisting of SEQ ID NO: 1, 2, 5, 9, and 10.

5. An immunogenic composition comprising an HLA class I B7-restricted HIV-1 polypeptide, said polypeptide comprising less than 50 amino acids in length, wherein the amino acid sequence of said polypeptide comprises an amino acid sequence selected from the group consisting of SEQ ID NO: 18, 25, and 26.

6. An immunogenic composition comprising an HLA class I B7-restricted HIV-1 polypeptide, wherein said polypeptide consists essentially of an amino acid sequence selected from the group consisting of SEQ ID NO: 18, 25 and 26.

7. The composition of claim 5, wherein said composition further comprises at least one polypeptide comprising an amino acid sequence selected from the group consisting of SEQ ID NO:16, 17, 19, 20, 21, 22, 23, and 24.

8. An immunogenic composition comprising an HLA class I Cw7-restricted HIV-1 polypeptide, said polypeptide comprising less than 50 amino acids in length, wherein the amino acid sequence of said polypeptide comprises the amino acid sequence of SEQ ID NO:27.

9. An immunogenic composition comprising a first polypeptide comprising an amino acid selected from the group consisting of SEQ ID NO: 3, 4, 6, 7, 8, 11, 12, 13, 14, 15, 18, 25, 26, and 27, and a second polypeptide comprising an amino acid selected from the group consisting of SEQ ID NO: 1, 2, 5, 9, 10, 16, 17, 19, 20, 21, 22, 23, and 24, wherein the length of said first and said second polypeptides is less than 50 amino acids.

10. A method of stimulating an HIV-specific immune response, comprising contacting an immune cell with an immunogen comprising a polypeptide, wherein said polypeptide is less than 50 amino acids in length and comprises an epitope selected from the group consisting of the amino acid sequence of SEQ ID NO: 3, 4, 6, 7, 8, 11, 12, 13, 14, 15, 18, 25, 26, and 27.

11. The method of claim 10, further comprising contacting said immune cell with a polypeptide comprises less than 50 amino acids and comprising an epitope selected from the group consisting of the amino acid sequence of SEQ ID NO: 1, 2, 5, 9, 10, 16, 17, 19, 20, 21, 22, 23, and 24.

12. The method of claim 11, wherein said immune cell is a CD8+ T cell.

13. The method of claim 11, wherein said T cell is contacted ex vivo.

14. A method of stimulating an HIV-specific immune response, comprising administering to a mammal an immunize comprising a peptide epitope selected from the group consisting of SEQ ID NO: 3, 4, 6, 7, 8, 11, 12, 13, 14, 15, 18, 25, 26, and 27, wherein said immune response comprises an increase in proliferation of HIV-specific CD8+ T cells.

15. The method of claim 14, wherein said mammal is a human.

16. The method of claim 15, wherein said human is a member of a Caucasian race.

17. The method of claim 15, wherein said human comprises an HLA allele selected from the group consisting of A3, B7, and Cw7.

18. A method of stimulating an HIV-specific immune response, comprising administering to a member of an asian race an immunogen comprising a peptide epitope selected from the group consisting of SEQ ID NO: 5, 6, 8, 10 and 11, wherein said immune response comprises an increase in proliferation of HIV-specific CD8+ T cells.

19. The method of claim 18, wherein said member comprises an HLA A11 allele.

20. A method for measuring an immune response in a patient, comprising
(a) providing a sample of cytotoxic T lymphocyte effector cells from a patient, said patient having been immunized with the composition of claim 1;
(b) providing HLA class I matched detectably-labeled target cells, said target cells having been pulsed with said polypeptide;
(c) contacting the effector cells and target cells; and
(d) determining the amount of label released by said target cells, wherein an increase in the amount of label released compared to a control value indicates the presence of an HIV-specific immune response in said patient.
